(19) United States
(12) Patent Application Publication
(10) Pub. No.: US 2023/0233143 A1
(43) Pub. Date: Jul. 27, 2023

(54) METHODS, SYSTEMS, AND APPARATUSES FOR THE DETECTION OF PAIN RELATED SYMPTOMS

(71) Applicant: University of Connecticut, Farmington, CT (US)

(72) Inventors: KI CHON, Mansfield, CT (US); YOUNGSUN KONG, Storrs, CT (US); HUGO POSADA-QUINTERO, Stafford Springs, CT (US)

(21) Appl. No.: 17/868,383

(22) Filed: Jul. 19, 2022

Related U.S. Application Data
(60) Provisional application No. 63/223,332, filed on Jul. 19, 2021.

(51) Int. Cl. A61B 5/00; A61B 5/0531

(52) U.S. Cl. CPC A61B 5/4824; A61B 5/0531; A61B 5/7278

(57) ABSTRACT

Methods, systems, and apparatuses for determining an indication of pain of a user are disclosed. Electrodermal activity (EDA) data from a sensor may be received. The EDA data may be indicative of one or more physiological signals derived from sweat gland activity of the user. Index values may be determined based on the EDA data. The index values may be compared to a threshold to determine if one or more of the values of the index value satisfies the threshold. Satisfying the threshold may indicate a pain in the user, a pain level for the user and/or a source of pain within the user. The indication may be caused to be output so that the user or others may be informed of an objective measurement of the pain in the user.

### CROSS-REFERENCE TO RELATED APPLICATIONS

This application claims the benefit of U.S. Provisional Application No. 63/223,332, filed Jul. 19, 2021, which is incorporated herein by reference in its entirety.

### GOVERNMENT LICENSE RIGHTS

This invention was made with government support under Grant No. N00014-19-1-2209 awarded by the Office of Naval Research. The government has certain rights in the invention.

### BACKGROUND

The assessment of pain in a patient is generally subjective and difficult to accurately discern. The assessment of pain may often be skewed due to personal characteristics of the patient and lack of efficient communication between a physician or other medication prescriber and the patient. Because of this, over-prescription of drugs and excessive drug use by the patient may occur. The excessive drug use by the patient may ultimately result in a physical or psychological drug dependency that is unhealthy for the patient.

### SUMMARY

It is to be understood that both the following general description and the following detailed description are exemplary and explanatory only and are not restrictive. Methods, systems, and apparatuses for the indication of pain associated with a user using electrodermal activity sensors are described herein. The present disclosure relates to a determination of when pain occurs, a pain level, and/or a source of the pain associated with the user.

In an example, the method may include receiving electrodermal activity (EDA) data of a user. The EDA data may be sent by a sensor and received by a computing device. An index value may be determined. For example, the index value may be determined based on the EDA data. An indication of pain in the user may be determined. The indication of pain for the user may be based on the index value. The indication of pain for the user may be output.

Additional advantages will be set forth, in part, in the description which follows or may be learned by practice. The advantages will be realized and attained by means of the elements and combinations particularly pointed out in the appended claims.

### BRIEF DESCRIPTION OF THE DRAWINGS

The accompanying drawings, which are incorporated in and constitute a part of this specification, together with the description, serve to explain the principles of the methods, systems, and apparatuses described herein:

FIG. 1 shows an example system;

FIG. 2 shows an example system;

FIG. 3 shows an example flowchart;

FIG. 4 shows an example flowchart;

FIGS. 5A and 5B show an example graphical recording;

FIG. 6 shows an example user interface;

FIG. 7 shows an example communication diagram;

FIG. 8 shows a flowchart of an example method; and

FIG. 9 shows a flowchart of an example method.

### DETAILED DESCRIPTION

As used in the specification and the appended claims, the singular forms “a,” “an,” and “the” include plural referents unless the context clearly dictates otherwise. Ranges may be expressed herein as from “about” one particular value, and/or to “about” another particular value. When such a range is expressed, another configuration includes from the one particular value and/or to the other particular value. Similarly, when values are expressed as approximations, by use of the antecedent “about,” it will be understood that the particular value forms another configuration. It will be further understood that the endpoints of each of the ranges are significant both in relation to the other endpoint, and independently of the other endpoint.

Throughout the description and claims of this specification, the word “comprise” and variations of the word, such as “comprising” and “comprises,” means “including but not limited to,” and is not intended to exclude, for example, other components, integers or steps. “Exemplary” means “an example of” and is not intended to convey an indication of a preferred or ideal configuration. “Such as” is not used in a restrictive sense, but for explanatory purposes.

It is understood that when combinations, subsets, interactions, groups, etc. of components are described that, while specific reference of each various individual and collective combinations and permutations of these may not be explicitly described, each is specifically contemplated and described herein. This applies to all parts of this application including, but not limited to, steps in described methods. Thus, if there are a variety of additional steps that may be performed it is understood that each of these additional steps may be performed with any specific configuration or combination of configurations of the described methods.

As will be appreciated by one skilled in the art, hardware, software, or a combination of software and hardware may be implemented to achieve the methods described herein. Furthermore, a computer program product on a computer-readable storage medium (e.g., non-transitory) having processor-executable instructions (e.g., computer software) embodied in the storage medium may also be implemented to process any of the methods described herein. Any suitable computer-readable storage medium may be utilized including hard disks, CD-ROMs, optical storage devices, magnetic storage devices, memristors, Non-Volatile Random Access Memory (NVRAM), flash memory, or a combination thereof.

Throughout this application reference is made to block diagrams and flowcharts. It will be understood that each block of the block diagrams and flowcharts, and combinations of blocks in the block diagrams and flowcharts, respectively, may be implemented by processor-executable instructions. These processor-executable instructions may be loaded onto a general purpose computer, special purpose computer, or other programmable data processing apparatus to produce a machine, such that the processor-executable instructions which execute on the computer or other programmable data processing apparatus create a device for implementing the functions specified in the flowchart block or blocks.

These processor-executable instructions may also be stored in a computer-readable memory that may direct a computer or other programmable data processing apparatus to function in a particular manner, such that the processor-executable instructions stored in the computer-readable memory produce an article of manufacture including processor-executable instructions for implementing the functions specified in the flowchart block or blocks. The processor-executable instructions may also be loaded onto a computer or other programmable data processing apparatus to cause a series of operational steps to be performed on the computer or other programmable apparatus to produce a computer-implemented process such that the processor-executable instructions that execute on the computer or other programmable apparatus provide steps for implementing the functions specified in the flowchart block or blocks.

Blocks of the block diagrams and flowcharts support a device or combinations of devices for performing the specified functions, combinations of steps for performing the specified functions, and program instruction means for performing the specified functions. It will also be understood that each block of the block diagrams and flowcharts, and combinations of blocks in the block diagrams and flowcharts, may be implemented by special purpose hardware-based computer systems that perform the specified functions or steps, or combinations of special purpose hardware and computer instructions.

Provided herein are methods, systems, and apparatuses for the detection of pain related symptoms using electrodermal activity (EDA) data from users. EDA is increasingly used as a measure of sympathetic function due to the accuracy with which EDA may be measured. In consideration of this, it may be beneficial to identify methods, systems, and/or apparatuses for using EDA to identify when pain occurs within a user (e.g., a patient, a drug-dependent individual, a health-compromised individual, or a communication-deficient being). In addition, it may be beneficial to identify methods, systems, and/or apparatuses to provide the user with an indication referencing a pain level of the user, for example.

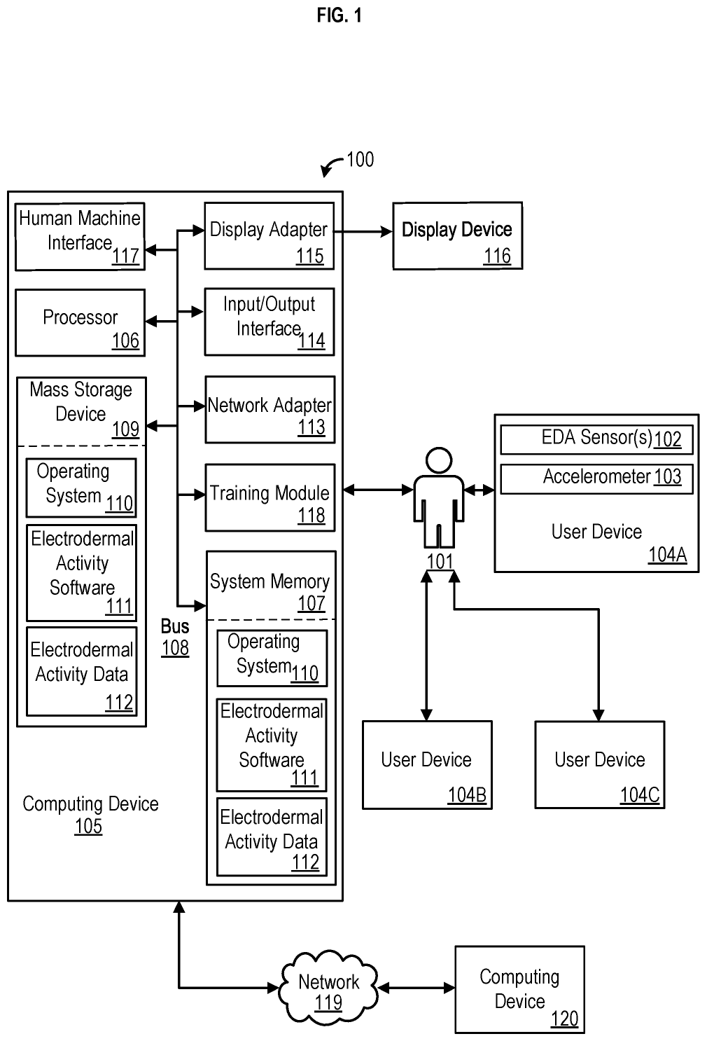

FIG. 1 shows an example system 100 for determining an indication of pain of a user (e.g., a user 101). For example, the system 100 may be representative of a system for the collection and evaluation of electrodermal activity (EDA) data of the user 101. The example system 100 may also be representative of a system for the collection and evaluation of other data (e.g., accelerometer data) associated with the user 101. Although only certain devices and/or components are shown, the system 100 may comprise a variety of other devices and/or components that support a wide variety of functions, such as network and/or communication functions. Those skilled in the art will appreciate that the present systems and methods may be used in various types of networks and systems that employ both digital and analog equipment.

The system 100 may include one or more sensors 102, 103 affixed to the user 101. For example, a plurality of sensors 102, 103 may be affixed to the user 101. For example, the plurality of sensors 102, 103 may be affixed to the user 101 via one or more user devices 104A-C, such as user device 104A. In another example, one or more sensors of the plurality of sensors 102, 103 may be affixed to different objects (e.g., different user devices 104A-C) associated with the user 101. For example, the user device 104A may be a wearable device, such as a wrist-worn adornment (e.g., a wristband, a smartwatch, or a diagnostic computer), a torso-worn adornment (e.g. a smart shirt or other device capable of positioning the sensors along a portion of the torso of the user 101), a facial-worn adornment, or the like. For example, each sensor of the plurality of sensors 102, 103 may be one or more of an EDA sensor 102, an accelerometer 103, or another form of sensor.

For example, each EDA sensor 102 may detect EDA data from the user 101. For example, each EDA sensor 102 may be configured to have all or at least a portion of the sensor positioned along the skin of the user 101. For example, each EDA sensor 102 may include one or more electrodes (not shown). For example, the electrodes may be stainless steel electrodes. In other examples, the electrodes may be made from another material. The electrodes may be communicably coupled to the EDA sensor 102 via one or more wires. For example, the electrodes and/or the EDA sensor 102 itself may be placed on one or more of the finger(s), scapula, sternum, instep, and/or another portion of the skin of the body of the user 101. For example, the user device 104A may include two EDA sensors 102, however fewer or greater numbers of EDA sensors 102 on the user device 104A or on other user devices (e.g., user devices 104B and/or 104C) is contemplated within the scope of this disclosure. For example, the EDA data may comprise or be derived from one or more physiological signals of the user 101. For example, the one or more physiological signals may be derived from sweat gland activity detected by the EDA sensor 102 along the skin of the user 101.

The system 100 may also include an accelerometer 103 affixed to the user 101. The accelerometer 103 may be affixed to any object associated with the user 101. For example, the accelerometer 103 may be part of the user device 104A, another user device 104B-C, or another wearable device attached to the user 101. For example, the accelerometer 103 may detect accelerometer data associated with the user 101. For example, the accelerometer 103 (or the user device 104A or wearable device containing the accelerometer 103) may send accelerometer data. For example, the accelerometer data may be sent to a computing device 105 and/or one or more of the user devices 104A-C via a wireless or wired internal or external transmission. The user device 104A, or another wearable device, may also include additional sensors, such as an electromyography sensor(s) and/or an electrocardiography sensor(s) that may each obtain associated data of the user 101.

The user device 104A may communicate with the computing device 105. The computing device 105 may be a diagnostic computer, a mobile phone, a smartphone, a tablet computer, a smartwatch, or similar device. The user device 104A may communicate via wired or wireless communication with the computing device 105. For example, the user device 104A may wirelessly communicate with the computing device 105 via a wireless communication signal using a wireless communication protocol (e.g., Bluetooth®, Bluetooth Low Energy (BLE), radio frequency (RF) (e.g., electromagnetic RF), WiFi, or any other known wireless communication protocol). For example, the user device 104A may send or transmit the EDA sensor data, the accelerometer data, and any other sensor data from the user device 104A to the computing device 105.

The computing device 105 may collect and send processed data derived from the EDA data received from the sensors (e.g., sensors 102, 103 and/or other sensors) to one or more external devices, such as user devices 104B and 104C, for example. As another example, the user devices 104A-C may each have a display. The user devices 104A-C may communicate (via wire(s) or wirelessly) directly with their respective displays. As another example, the computing device 105 may communicate directly with the display of one or more of the user devices 104A-C.

The system 100 may comprise a second computing device 120. The second computing device 120 may comprise one or more of a client device, a personal computer, computing station, workstation, portable computer, laptop computer, mobile phone, tablet device, smartphone, smartwatch, or activity tracker. For example, the second computing device 120 may be associated with a physician, nurse, nurse practitioner, clinic, hospital, doctor's office, or other medical personnel or medical facilities associated with caring for or monitoring the health welfare of the user 101. For example, the computing device 105 may communicate directly or indirectly with the second computing device 120 via a network (e.g., network 119). For example, the computing device 105 and/or the user device 104A may send EDA data and index values determined from EDA data for the user 101 to the second computing device 120 via the network 119. For example, the computing device 105 and/or the user device 104A may send an indication, or a signal referencing the indication of pain of the user 101 (e.g., the user 101 is experiencing pain, the level of pain experienced by the user 101, and/or the source of the pain in the user 101) to the second computing device 120 via the network 119.

The system 100 may comprise one or more networks, such as the network 119. The network 119 may provide a communication path between the computing device 105, the second computing device 120, and/or one or more of the user devices 104A-C. The network 119 may be an optical fiber network, a coaxial cable network, a hybrid fiber-coaxial network, a wireless network, a satellite system, a direct broadcast system, an Ethernet network, a high-definition multimedia interface network, a Universal Serial Bus (USB) network, or any combination thereof. Data may be sent on the network 119 via a variety of transmission paths, including wireless paths (e.g., satellite paths, Wi-Fi paths, cellular paths, etc.) and wired paths (e.g., wired paths, a direct feed source via a direct line, etc.). The network 119 may comprise public networks, private networks, wide area networks (e.g., Internet), local area networks, and/or the like. The network 119 may be configured to receive and evaluate EDA data and/or index values from a variety of sources using a variety of network paths, protocols, devices, and/or the like. The network 119 may deliver EDA data, index values, user interface commands, and/or indications of pain in a user 101 from the computing device 105 or user device 104A to one or more other computing devices (e.g., the second computing device 120, user devices 104B-C, etc.).

For example, the user devices 104A-C may receive (e.g., from the user devices 104A-C and/or the computing device 105) an indication to be displayed to the user 101. The indication may be text, one or more colors of lights, a sound, and/or a vibration, for example. As an example, the indication may reference a level of pain for the user 101. For example, the indication may correlatively intensify based on an increase in the level of pain of the user 101 (e.g., the frequency of vibration may increase based on the level of pain of the user 101 increasing). As a further example, the indication may provide an index to inform the user 101 of the pain level associated with the user 101.

The computing device 105 may comprise one or more processors 106 or processing units, a system memory 107, and a system bus 108 that couples various system components of the computing device 105, including the processor 106 to the system memory 107. In the case of multiple processors 106, the system may utilize parallel computing.

The system bus 108 represents one or more of several possible types of bus structures, including a memory bus or memory controller, a peripheral bus, an accelerated graphics port, and a processor or local bus using any of a variety of bus architectures. By way of example, such architectures may comprise an Industry Standard Architecture (ISA) bus, a Micro Channel Architecture (MCA) bus, an Enhanced ISA (EISA) bus, a Video Electronics Standards Association (VESA) local bus, an Accelerated Graphics Port (AGP) bus, a Peripheral Component Interconnects (PCI), a PCI-Express bus (USB) and the like.

The system bus 108 may also be implemented over a wired or wireless network connection to each of the subsystems, including the processor 106, a mass storage device 109, an operating system 110, EDA software 111, EDA data 112, a network adapter 113, an Input/Output (I/O) interface 114, a display adapter 115, a display device 116, a human machine interface 117, and a training module 118. It is understood that the system bus 108 and each of the aforementioned subsystems may be contained within each of the user devices 104A-C at physically separate locations, connected through buses of this form; in effect implementing a fully distributed system.

The computing device 105 may operate on and/or comprise a variety of computer-readable media (e.g., non-transitory computer-readable media). Computer-readable media may be any available media that is accessible by the computing device 105 and comprises both volatile and non-volatile media and removable and non-removable media. The system memory 107 may comprise computer-readable media in the form of volatile memory and removable and non-removable media. The system memory 107 may comprise computer-readable media in the form of volatile memory, such as random access memory (RAM), and/or non-volatile memory, such as read only memory (ROM). The system memory 107 typically contains data and/or program modules, such as an operating system 110 and EDA software 111 that are accessible to and/or are operated on by the one or more processors 106.

The training module 118 may be configured to use machine learning techniques to train, based on an analysis of one or more training datasets of a plurality of training data sets (e.g., a plurality of training data sets 205) at least one machine learning-based classifier (e.g., a machine learning-based classifier 210). For example, the EDA software 111 may be configured to include machine learning aspects that may aid the training module 118 to analyze the one or more training datasets.

The EDA software 111 may be configured to receive the one or more physiological signals from the one or more sensors (e.g., the sensors 102, 103 and any other sensors associated with the user 101). The EDA software 111 may interpret the physiological signals into data (e.g., EDA data, and/or index values of the EDA data). For example, the data may be index values determined by the EDA software 111. As another example, the EDA software 111 may determine a pain level of the user 101 based on the index values.

The system memory 107 may also contain accelerometer software (not shown). For example, the accelerometer software may remove motion artifacts from the physiological signals/data. It is understood that motion artifacts may be associated with a voluntary and/or involuntary movement of the user 101. The motion artifacts in the physiological signals may cause erroneous EDA data and correspondingly erroneous index values that may ultimately cause an inaccurate indication of pain in the user 101. The removal of motion artifacts may be beneficial to mitigate the risk against false readings that may result in an incorrect indication of pain and/or pain level of the user 101.

The computing device 105 may also comprise other removable/non-removable, volatile/non-volatile computer storage media. By way of example, the mass storage device 109 may provide non-volatile storage of computer code, computer readable (e.g., processor-executable) instructions, data structures, program modules, and other data for the user device 105. For example, the mass storage device 109 may be a hard disk, a removable magnetic disk, a removable optical disk, a removable magnetic disk, a removable optical disk, magnetic cassettes or other magnetic storage devices, flash memory cards, CD-ROM, digital versatile disks (DVD) or other optical storage, random access memory (RAM), read only memory (ROM), electrically erasable programmable read-only memory (EEPROM), and the like.

Any number of program modules may be stored on the mass storage device 109, including by way of example, the operating system 110, EDA software 111, and EDA data 112. EDA data 112 may include the one or more physiological signals (e.g., the EDA data) of the user 101. The EDA data 112 may also include index values and/or accelerometer data for the user 101.

The display device 116 may also be connected to the system bus 108 via an interface, such as the display adapter 115. It is contemplated that the computing device 105 may have more than one display adapter 115 and the computing device 105 may have more than one display device 116. For example, a display device 116 may be a monitor, an LCD (Liquid Crystal Display), light emitting diode (LED) display, smart glass, or a projector. In addition to the display device 116, other output peripheral devices may comprise components, such as speakers (not shown) and a printer (not shown) which may be connected to the computing device 105 via the Input/Output interface 114. Any step and/or result of the methods may be output in any form to an output device. Such output may be any form of visual representation, including, but not limited to, textual, graphical, animation, audio, tactile, and the like. The display device 116 and the computing device 105 may be part of one device, or separate devices.

For purposes of illustration, application programs and other executable program components, such as the operating system 110 are illustrated herein as discrete blocks, although it is recognized that such programs and components reside at various times in different storage components of the computing device 105, and are executed by the data processor(s) of the computing device 105. An implementation of the EDA software 111 and/or the EDA data 112 may be stored on or transmitted across some form of computer-readable media. Any of the disclosed methods may be performed by computer-readable, processor-executable instructions embodied on computer-readable media. In addition, any of the components or functions of the computing device 105 may equally be a part of and implemented in any one or each of the user devices 104A-C.

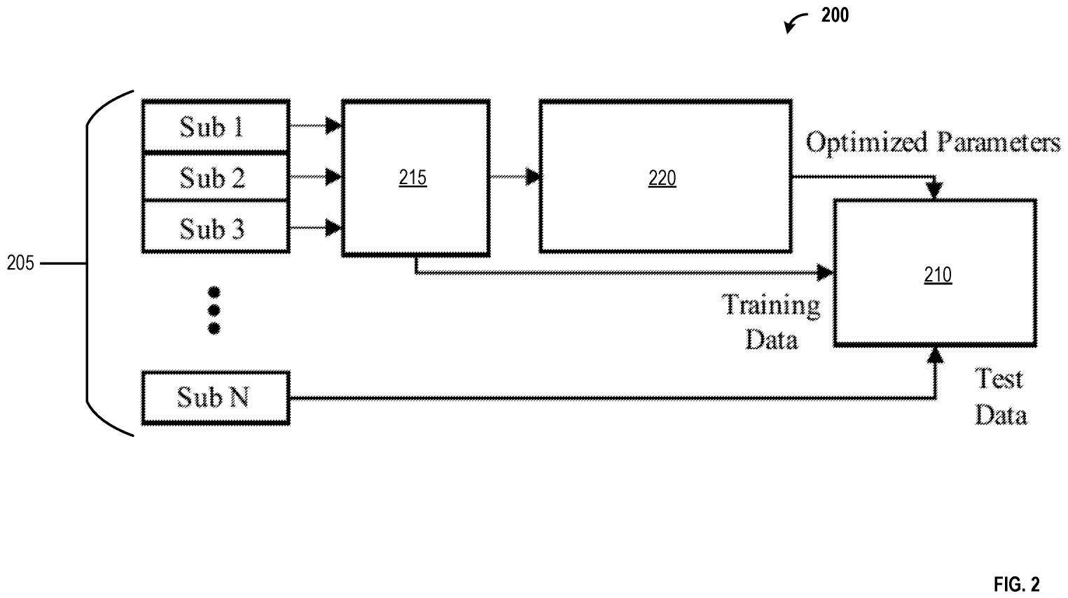

FIG. 2 shows an example system 200 configured to use machine learning techniques to train at least one machine learning-based classifier 210. For example, the system 200 may be part of one or more of the computing device 105 or the user devices 104A-C. The system 200 may train the at least one machine learning-based classifier based on an analysis of one or more training data sets 205 by a training module (e.g., the training module 118). The at least one machine learning-based classifier 210, once trained, may be configured to classify changes in accuracy, sensitivity, and/or specificity associated with measurements of pain at a specific pain level. The training module 215 may send one more trained data sets to the classifier 210 via a grid search computational unit 220. For example, the grid search computational unit 220 may implement a grid search technique in an analysis of the one or more trained data sets. The grid search computational unit 220 may identify learning parameters associated with the trained data sets via the grid search technique, for example. The grid search computational unit 220 may compute and/or optimize the identified parameters based on the one or more trained data sets. For example, the grid search computational unit 220 may send the optimized parameters (e.g., criterion, activation, and/or learning rate) associated with the analysis of the one or more trained data sets to the classifier 210. As another example, the training module 215 may send training data directly to the classifier 210. As another example, historical data of recorded pain level measurements associated with one or more users may aid in the development in a definition of an objective pain measurement, or score or level of pain for any specific ailment. For example, a mean is computed across a plurality of users experiencing a similar ailment. The mean may indicate an objective score referencing a pain level. As another example, the mean may be a dynamic variable that may change based on increased readings of various other users, over time.

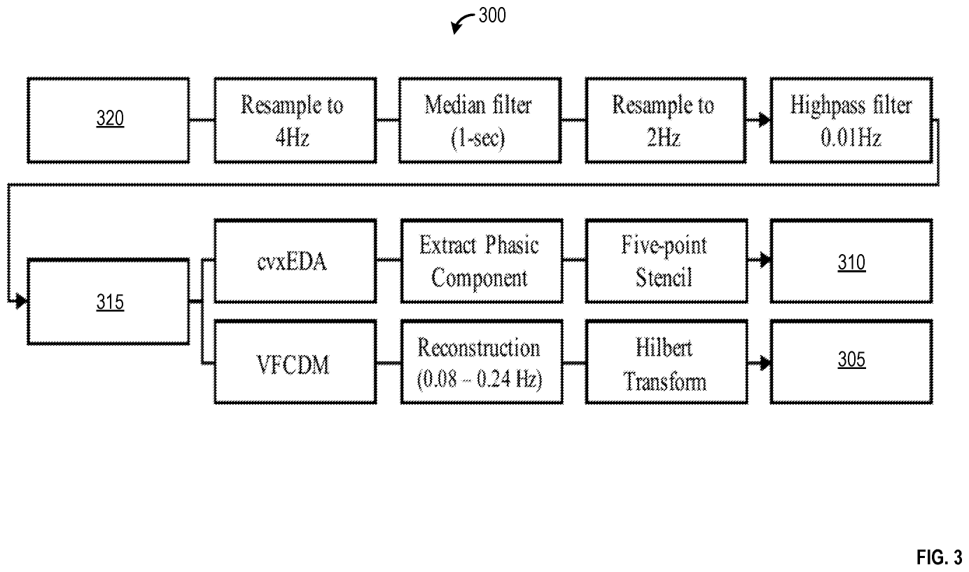

FIG. 3 shows a flowchart of an example method 300 for determining a time-varying index of sympathetic activity (TVSymp) value 305 and a derivative of phasic component of EDA (dPhEDA) value 310. The method 300 may be performed by the computing device 105 and/or any of the user devices 104A-C. For example, each of the TVSymp value 305 and the dPhEDA value 310 may be determined based on processed EDA data 315.

The processed EDA data 315 may be determined based on EDA data 320. For example, the EDA data 320 may be initially recorded at 130 Hz. In other examples, the EDA data may be processed at any other frequency. The initially recorded EDA data 320 may be resampled. For example, the EDA data 320 may be resampled to 4 Hz, however, in other examples the EDA data may be resampled at another frequency. A median filter may be applied to the resampled EDA data 320. For example, the median filter may have a one-second window. In other examples the median filter may have a window of less than or greater than 1 second. The EDA data 320 may be resampled to 2 Hz, however, in other examples the EDA data may be resampled at another frequency. For example, the EDA data 320 may be resampled to 2 Hz after the EDA data 320 has the median filter applied. A highpass filter may be applied to the resampled EDA data 320. For example, the highpass filter may have a cutoff frequency at 0.01 Hz, however, in other examples the cutoff frequency be at any other frequency. For example, the EDA data 320 having had the highpass filter applied, may result in the processed EDA data 315.

The TVSymp value 305 may be determined by implementing a variable frequency complex demodulation (VFCDM). For example, the VFCDM may be used to reconstruct the processed EDA data 315 with components in the range of 0.08-0.24 Hz. An instantaneous amplitude of the reconstructed EDA data 315 may be obtained using a Hilbert transform, for example. The TVSymp value 305 may be computed based on the VFCDM and the instantaneous amplitude. For example, the TVSymp value 305 may be computed as follows:

$$Z ( t ) = {X}^{′} ( t ) + {iY}^{′} ( t ) = a ( t ) {e}^{j θ ( t )}$$

$$a ( t ) = {[ {X}^{′2} ( t ) + {Y}^{′2} ( t ) ]}^{\frac{1}{2}}$$

$$θ ( t ) = arc tan ( {Y}^{′} ( t ) / {X}^{′} ( t ) )$$

where Z(t) may be an analytic signal. Additionally, a(t) may be the TVSymp value and may be obtained by computing the instantaneous amplitude of Z(t), for example. Also, X′(t) and Y′(t) may form a complex conjugate pair.

A modified TVSymp (MTVSymp) value may be determined based on the TVSymp value. For example, the MTVSymp value may be determined so that EDA changes caused by pain may be emphasized and so that baseline EDA responses from prior EDA segments may be removed. For example, the MTVSymp value may be computed as follows:

$$MTV {Symp}_{t} = \{ \begin{matrix}{a}_{t} - {μ}_{t} , & {μ}_{t} \leq {a}_{t} \\ 0 , & {μ}_{t} > {a}_{t}\end{matrix} ,$$

$${μ}_{t} = \frac{1}{k \cdot {F}_{s}} {\sum }_{i = t - k \cdot {F}_{s}}^{t - 1} {a}_{i}$$

where k may be a length of time window (e.g., 5 seconds), at may be a time point ‘t’ of TVSymp, aᵢ may be a time point T of TVSymp, μₜ may be an average of a plurality of TVSymp samples, and Fₛ may be a sampling frequency (e.g., 2 Hz). μₜ may correspond to k-seconds associated with time point ‘t,’ for example. For example, each time point of MTVSymp may be computed by subtracting an average of samples corresponding to k-seconds back each time point associated with TVSymp, and setting the MTVSymp to zero in the case wherein the averaged value may be greater than TVSymp.

The dPhEDA value 310 may comprise tonic and/or phasic components, for example. The dPhEDA value 310 may be determined by implementing a convex optimization approach (cvxEDA) so that the one or more processed EDA data 315 may be decomposed into phasic and/or tonic components. The phasic components of the processed EDA data may then be used going forward and the tonic components discarded. A five-point stencil central finite differences equation may then be utilized to determine the dPhEDA value 310, for example, as follows:

$$d P h E D {A}_{n} = \frac{{x}_{{phasic}_{n - 2}} - 8 \cdot {x}_{{phasic}_{n - 1}} + 8 \cdot {x}_{{phasic}_{n + 1}} - {x}_{{phasic}_{n + 2}}}{12 \cdot ( 1 / {F}_{s} )} ,$$

where x_{phasic} may be a processed phasic component extracted from the processed EDA data 315 and Fₛ may be a sampling frequency (e.g., 2 Hz).

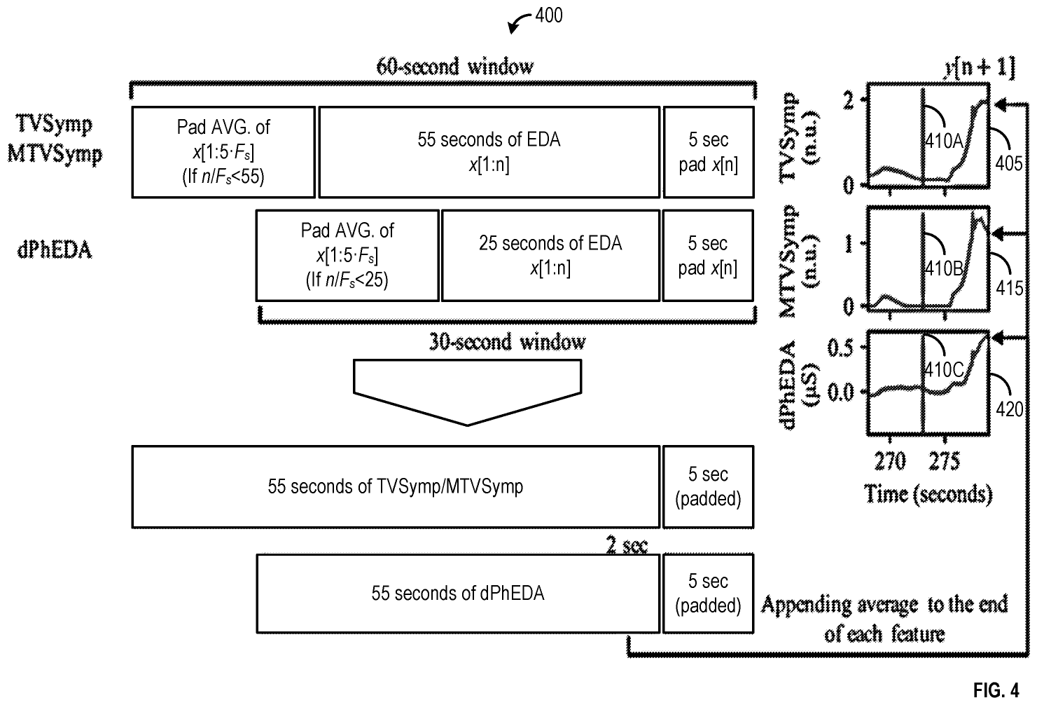

FIG. 4 shows an example flowchart 400 of a computation of each of the TVSymp value (e.g., the TVSymp value 305), the MTVSymp value, and the dPhEDA value (e.g., the dPhEDA value 310). For example, the example flowchart 400 may be representative of an example scheme wherein a time series for real-time monitoring may apply. As another example, the TVsymp value and the MTVSymp value may be considered under a time window (e.g., a 60 second time window). For example, a determination may be made with regard to an ideal time period that the time window may be padded. For example, the time window may be padded by a time period of five seconds. In other examples, the time window may be padded by a time period greater than or less than five seconds, such as anywhere between 0-20 seconds within a 60 second time window. A last value of EDA data may be additionally padded at a multiplicative value (e.g., 20 times or 5 seconds multiplied by 4 Hz) at an end of each signal so that a corruption of the TVSymp/MTVSymp values may be avoided. The time series associated with each of the TVSymp/MTVSymp values may be averaged to compute features associated with the TVSymp/MTVSymp values. For example, the last two seconds of the time series associated with each of the TVSymp/MTVSymp values may be averaged. The average of each of the TVSymp/MTVSymp values may be appended to the end of the computed real-time features, for example. An example of a resultant time series of TVSymp values is represented in an example graphical representation 405, wherein a stimulus to generate pain in a user (e.g., user 101) is represented by vertical line 410A. The example graphical representation 405 is represented by time along the x-axis and TVSymp values along the y-axis, for example. An example of a resultant time series of MTVSymp values is represented in an example graphical representation 415, wherein a stimulus to generate pain in the user 101 is represented by vertical line 410B. The example graphical representation 415 is represented by time along the x-axis and MTVSymp values along the y-axis, for example.

For example, the dPhEDA value may be considered under a time window (e.g., a 30 second time window). For example, a determination may be made with regard to an ideal time period that the time window may be padded. For example, the time window may be padded by a time period of five seconds. In other examples, the time window may be padded by a time period greater than or less than five seconds, such as anywhere between 0-15 seconds within a 30 second time window. A last value of EDA data may be additionally padded at a multiplicative value (e.g., 20 times or 5 seconds multiplied by 4 Hz) at an end of each signal so that a corruption of the dPhEDA value may be avoided. The time series associated with the dPhEDA value may be averaged to compute features associated with the dPhEDA value. For example, the last two seconds of the time series associated with the dPhEDA value may be averaged. The average of the dPhEDA value may be appended to the end of the computed real-time features, for example. An example of a resultant time series of dPhEDA values is represented in an example graphical representation 420, wherein a stimulus to generate pain in the user 101 is represented by vertical line 410C. The example graphical representation 420 is represented by time along the x-axis and dPhEDA values along the y-axis, for example.

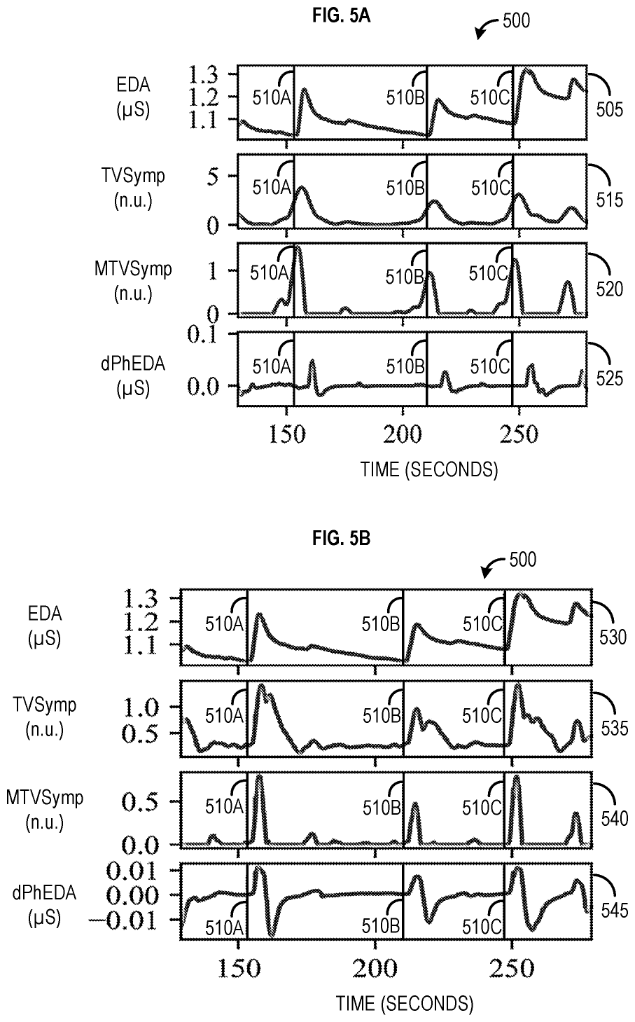

FIGS. 5A and 5B show example graphical representations 500 of collected EDA data and the index values derived from that EDA data. For example, the EDA data may be collected from the one or more EDA sensors 102 of FIG. 1. For example, FIG. 5A may show collected EDA data that represents at least a portion of a recording of EDA data for the user 101 as well as index values (e.g., TVSymp, MTVSymp, and dPhEDA) derived from that EDA data. For example, the EDA data collected from the one or more sensors 102 for the user 101 is shown in graphical representation 505, where stimuli provided to the user 101 to induce pain in the user 101 is represented by vertical lines 510A-C. For example, the index value TVSymp data, determined from the EDA data in graph 505, is shown in graphical representation 515, wherein stimuli provided to the user 101 to induce pain in the user 101 is represented by the vertical lines 510A-C. For example, the index value MTVSymp data, determined from the EDA data in graph 505, is shown in graphical representation 520, wherein stimuli provided to the user 101 to induce pain in the user 101 is represented by the vertical lines 510A-C. For example, the index value dPhEDA data, determined from the EDA data in graph 505, is shown in graphical representation 525, wherein stimuli provided to the user 101 to induce pain in the user 101 is represented by the vertical lines 510A-C.

FIG. 5B shows collected EDA data that may represent a recording of EDA data from the one or more sensors 102 for the user 101 and the index values derived from the EDA data and manipulated by the aforementioned process described in FIG. 4 (e.g., windowing). For example, the EDA data collected from the one or more sensors 102 for the user 101 is shown in graphical representation 530, wherein stimuli provided to the user 101 to induce pain in the user 101 is represented by the vertical lines 510A-C. For example, the index value TVSymp data, determined from the EDA data in graph 505, is shown in graphical representation 535, wherein stimuli provided to the user 101 to induce pain in the user 101 is represented by the vertical lines 510A-C. For example, the index value MTVSymp data, determined from the EDA data in graph 505, is shown in graphical representation 540, wherein stimuli provided to the user 101 to induce pain in the user 101 is represented by the vertical lines 510A-C. For example, the index value dPhEDA data, determined from the EDA data in graph 505, is shown in graphical representation 545, wherein stimuli provided to the user 101 to induce pain in the user 101 is represented by the vertical lines 510A-C.

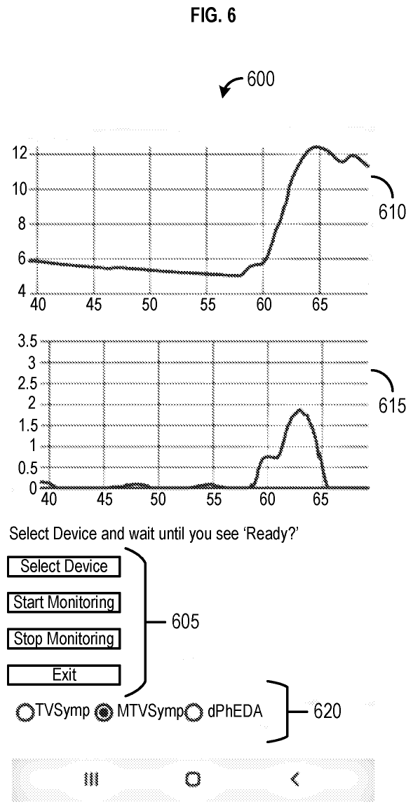

FIG. 6 shows an example user interface (UI) 600 for an EDA data processing application. For example, the EDA data processing application may be a part of or accessible by and used by the computing device 105, the user device 104A or any other device described herein. In addition, the UI 600 may be displayed on a display associated with the computing device 105 (e.g., the display device 116), a display associated with the user device 104A, or a display associated with any other device described herein. For example, the EDA data processing application may process EDA data received by the one or more EDA sensors 102. The EDA data processing application may be configured to collect and/or process EDA data in real-time. For example, the EDA data processing application may collect and/or process EDA data by being included on and/or communicating with the user device 104A described in FIG. 1.

The UI 600 may provide a user (e.g., the user 101) with options that the user may select in response to a prompt. For example, the prompt may indicate that the EDA data processing application is ready to process a request 605. As an example, the request 605 may be to select a device (e.g., the user device 104A), start monitoring EDA data, stop monitoring EDA data, or to exit. However, additional and/or different requests may be included in the request 605 on the UI 600.

The processing application may receive raw EDA data. For example, the processing application may receive the EDA data (e.g., raw EDA data) from the one or more EDA sensors 102 and/or the user device 104A. The UI 600 may display the EDA data in a graphical format on a first portion 610 of the UI 600. The EDA data processing application may implement various algorithms to display index values, such as TVSymp, MTVSymp, or dPhEDA determined based on the EDA data as well. For example, the UI 600 may display the determined TVSymp, MTVSymp, and/or the dPhEDA data in a graphical format on a second portion 615 of the UI 600.

The UI 600 may also provide the user with an option to select which index value or values to display in the second portion 615 of the UI 600. For example, the UI 600 may allow for the user to select (e.g., at a selection portion 620 of the UI 600) TVSymp, MTVSymp, or dPhEDA) (e.g., index values) as the index value to determine and display based on the EDA data. As an example, the processing application may implement any algorithm to determine the one or more index values based on the EDA data. While the example second portion 615 of the UI 600 of FIG. 6 only shows the graphical representation of one of the index values, in other examples more than one and/or all of the index values may be shown concurrently on the second portion 615.

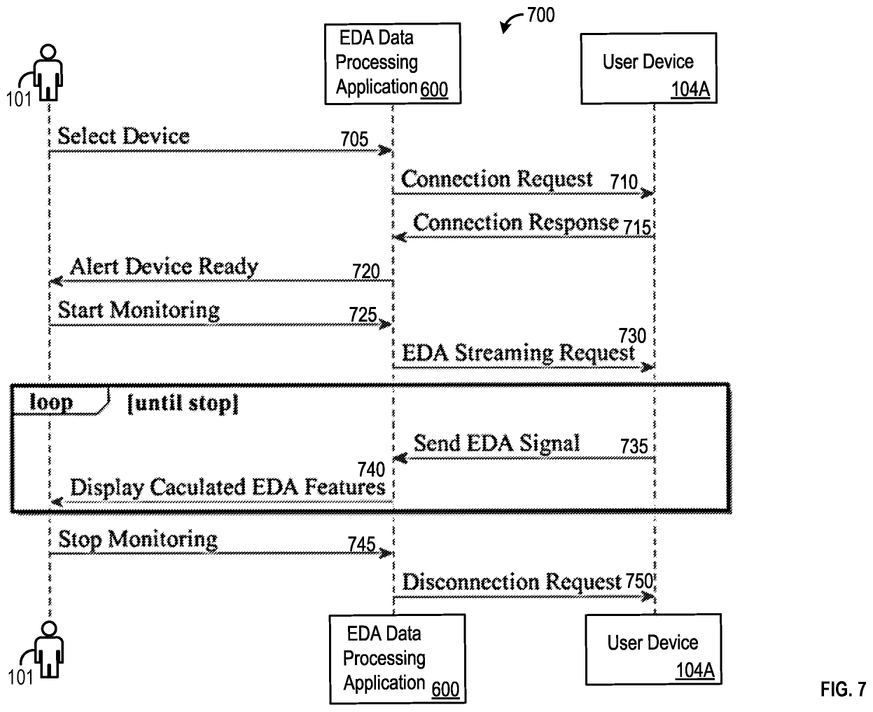

FIG. 7 shows an example communication diagram 700 for communication of EDA data with the UI 600 of the EDA data processing application of FIG. 6. At 705, a user (e.g., the user 101) may interact with the EDA processing application by selecting a device via a first request (e.g., the request 605) in the UI 600. For example, the device selected may comprise the user device 104A or any other device (e.g., a wearable device) comprising the one or more EDA sensor(s) 102. For example, the device selected may be the same device upon which the UI 600 is being presented and upon which the EDA data processing application is operating. At 710, the EDA data processing application may send a connection request to the selected device (e.g., a wearable device, such as the user device 104A) associated with the user. The connection request may be sent via a wired or wireless communication. In certain examples, the sending of the connection request may be an internal transmission within a device, such as the user device 104A. For example, the connection request may be sent based on the EDA data processing application's receipt of the user's selection of the device via the UI 600.

At 715, the selected device may send a connection response to the EDA data processing application. The connection response may be sent via a wired or wireless communication. In certain examples, the sending of the connection response may be an internal transmission within a device, such as the user device 104A. For example, the selected device may send the connection response based on receipt of the connection request. At 720, the EDA data processing application may indicate to the user that the selected device is ready to begin collecting EDA data from the user. For example, the EDA data processing application may indicate the selected device is ready via a message on the UI 600. For example, the indication may be displayed on a display device associated with the computing device on which the EDA data processing application is operating (e.g., the display 116 of the computing device 105 or a display associated with the user device 104A). For example, the application may display or present the alert to the user based on receipt of the connection response.

At 725, the user may interact with the EDA data processing application by inputting a second command. For example, the input associated with the second command may indicate, via the UI 600, to the EDA data processing application to start monitoring (e.g., receiving) EDA data from the one or more EDA sensor(s) 102. At 730, the EDA data processing application may send an EDA streaming request to the selected device. The EDA streaming request may be sent via a wired or wireless communication. In certain examples, the sending of the EDA streaming request may be an internal transmission within a device, such as the user device 104A. For example, the EDA data processing application may send the EDA streaming request based on receipt of the input of the user's second command.

At 735, the selected device may send EDA data to the EDA data processing application. The EDA data may be sent via a wired or wireless communication. In certain examples, the sending of the EDA data may be an internal transmission within a device, such as the user device 104A. For example, the selected device may send the EDA data based on receipt of the EDA streaming request. The EDA data processing application may receive the EDA data, process the EDA data, and determine associated index values (e.g., TVSymp, MTVSymp, and/or dPhEDA) based on the EDA data. At 740, the EDA data processing application may cause the received EDA data and/or one or more of the determined index values to be displayed to the user. The sending of the EDA data and the display of the EDA data and/or determined index values may be a recurring loop continuously occurring until a timer lapses or a command is received to stop monitoring the EDA data for the user.

At 745, a command may be input by the user for the EDA data processing application to stop monitoring the EDA data for the user. For example, the command may be input by the user via the UI 600. At 750, the EDA data processing application may send a disconnection request to the selected device. The disconnection request may be sent via a wired or wireless communication. In certain examples, the sending of the disconnection request may be an internal transmission within a device, such as the user device 104A. For example, the EDA data processing application may send the disconnection request based on receipt of the user's command input via the UI 600.

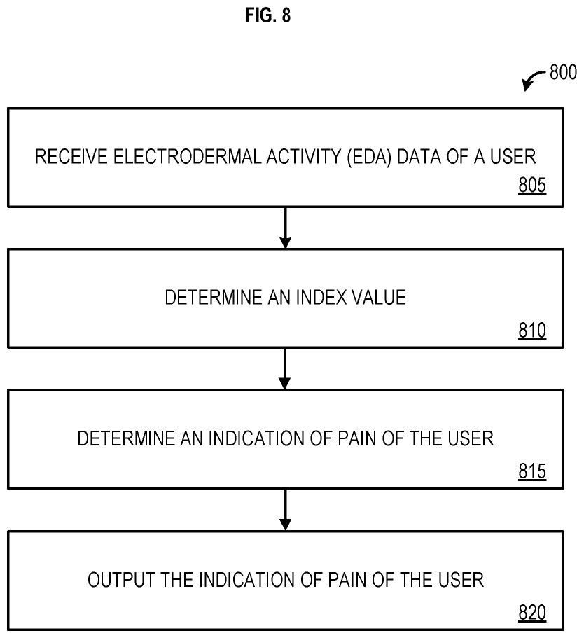

FIG. 8 shows a flowchart of an example method 800 for the indication of pain of a user (e.g., a patient, a drug dependent individual, a health-compromised individual, or a communication-deficient being). The method 800 may be performed by the computing device 105 and/or any of the user devices 104A-C.

At step 805, EDA data of a user (e.g., the user 101) may be received. For example, the EDA data may be received by the computing device 105, the user device 104A or any other device described herein. For example, the EDA data may be received from one or more sensors, such as the EDA sensor(s) 102 of FIG. 1. For example, the EDA data may be received while the user 101 is experiencing varying instances of pain. For example, EDA data received from the user 101 may be based on varying stimuli associated with pain. As an example, the EDA data may be continuously received in the instance wherein the user 101 selects a constant EDA monitoring option of the user's 101 body (e.g., a smart watch or an at-home patient monitoring device). As an example, the EDA data may be or may be associated with information that may be indicative of one or more physiological signals of the user 101. The one or more physiological signals may be derived from sweat gland activity of the user 101 and received from the EDA sensor(s) 102, or probes associated with the sensors 102, contacting the skin of the user 101.

For example, other sensor data associated with the user 101 may be received. The other sensor data may include one or more of electromyographical data readings of the user 101, electrocardiographical data readings of the user 101, and/or accelerometer data readings associated with movement of the user 101. The electromyographical data readings may be received from one or more electromyography sensors, the electrocardiographical data readings may be received from one or more electrocardiography sensors, and the accelerometer data may be received from an accelerometer 103. For example, the accelerometer 103 may be a tri-axial accelerometer. For example, the accelerometer may determine movement of the user 101. For example, the movement data of the user 101 may be used to determine portions of the EDA data which may not be desirable to use due to the potential for the motion of the user 101 causing erroneous index values (e.g., TVSymp, MTVSymp, or dPhEDA). Each of the electromyography sensor, the electrocardiography sensor, and the accelerometer 103 may be included with the same device (e.g., the user device 104A) as the one or more EDA sensors 102 or may be part of and received by another device, such as another wearable device or another one of the user devices 104B-C. Each of the EDA data, the accelerometer data, the electromyographical data, and the electrocardiographical data may be received via a wired or wireless (e.g., Bluetooth®, BLE, RF (e.g., electromagnetic RF), WiFi, or any other known wireless communication protocol) communication. For example, EDA data and any of the other data may be sent by the user device 104A. For example, the EDA data and the other data may be sent by the user device 104A as part of an internal transmission from the respective sensors to another portion of the user device 104A. In another example, the user device 104A may send the EDA data and the other sensor data to the computing device 105 or another user device 104B-C via the wired or wireless communication.

A determination may be made as to whether all or portions of the received EDA data is valid. For example, the determination may be made by the computing device 105, the user device 104A or any other device described herein. For example, EDA data received while the user 101 is performing a certain amount of movement or exertion may not be useful in the analysis. For example, accelerometer data may be received from an accelerometer 103. For example, the accelerometer may be associated with the movement of the user 101. The accelerometer data may be compared to a motion threshold. A determination may be made as to if all or portions of the accelerometer data satisfy (e.g., is less than or less than or equal to or is greater than or greater than or equal to) the motion threshold. For example, if a first portion of the accelerometer data does not satisfy (e.g., the accelerometer data is less than or less than or equal to) the motion threshold, a determination may be made that the EDA data associated with (e.g., collected at the same time as) the first portion of the accelerometer data is valid EDA data. For example, if a second portion of the accelerometer data satisfies (e.g., the accelerometer data is greater than or greater than or equal to) the motion threshold, a determination may be made that the EDA data associated with the second portion of the accelerometer data is invalid EDA data. For example, satisfying the motion threshold may be switched from greater than or greater than or equal to, to less than or less than or equal to and the determination of which EDA data is valid and invalid may be similarly inverted (e.g., valid data satisfies the threshold and invalid data does not satisfy the threshold) in certain examples. For example, the second portion of the EDA data comprising invalid data may be removed from further analysis and the first portion of the EDA data comprising valid data may be further analyzed and processed.

At step 810, an index value may be determined. For example, the index value may be determined by the computing device 105, the user device 104A or any other device described herein. For example, the index value may be determined based on the EDA data. For example, the index value may be determined based on the valid EDA data (e.g., the first portion of the EDA data). For example, the index value may be one or more of TVSymp of the EDA data, MTVSymp of the EDA data, or dPhEDA data. The index value may be determined using a frequency complex demodulation, for example For example, the one or more index values may serve as a basis by which pain occurring in the user 101 is determined.

At step 815, an indication of pain in the user 101 may be determined. For example, the determination may be made by the computing device 105, the user device 104A or any other device described herein. For example, the determination of the indication of pain in the user 101 may be based on the index value. For example, the determination of pain in the user may be based on a value of the index value satisfying a threshold. For example, a plurality of values (e.g., data points) for one of the index values (e.g., TVSymp, MTVSymp, dPhEDA) may be determined from the EDA data of the user 101. Each of the plurality of values of the particular index value may be compared to the threshold value. A determination may be made, based on the comparison, whether the particular value of the index value satisfies (e.g., is greater than or greater than or equal to) the threshold. If the value of the index value satisfies the threshold it may indicate pain is occurring in the user 101. If the index value does not satisfy the threshold, it may indicate no pain or no significant pain is occurring in the user 101.

For example, a change in the index value over time may indicate pain in the user 101. For example, a second value for the particular index value may be derived from a portion of the EDA data. For example, the second value may be determined based on the EDA data. As another example, the second value for the particular index value may precede the second portion of the EDA data that the value for the particular index value may be derived from. For example, whether a difference between the index value and the second index value satisfies a threshold may be determined. For example, the value and the second value may be compared to determine a difference between the value and the second value. The difference may then be compared to the threshold as described above to determine an indication of pain in the user 101. For example, the indication of pain for the user 101 may be based on the difference satisfying the threshold.

A pain level of the user 101 may be determined. For example, the determination of the pain level of the user 101 may be based on the index value. For example, differing levels or amounts of the particular index value may be associated with a particular pain index or score. Based on the level of the value for the particular index value, the level of pain (e.g., the pain index or score) for the user 101 may be determined. For example, multiple pain thresholds may be provided, each indicating a particular score or level of pain in the user 101. For example, the values for the index value may be compared to each of these pain thresholds to determine the pain level or score for the user 101. For example, the difference of the values of the index value may also indicate the level of pain in the user 101. For example, the difference of the values of the particular index value may be determined as described above. The difference may then be compared to multiple thresholds as described above to determine a pain score of level of pain in the user 101. For example, the pain level for the user 101 may be based on the difference satisfying one or more of the plurality of thresholds.

In addition, a source or location of the pain associated with the user 101 may be determined. For example, the determination of the source or location of pain on the user 101 may be based on the index value (e.g., the amount of the index value, the number of peaks of the index value over a period of time, etc.).

The threshold(s) may be static (e.g., a predetermined or preset threshold that is not adjustable) or dynamic. For example, the dynamic threshold may vary and may be determined based on one or more of historical EDA data for the user 101, historical index values for the user 101, historical EDA data for all users, or historical index values for all users. For example, the dynamic threshold may be based on the historical data of the user 101. For example, satisfying the threshold may be switched from greater than or greater than or equal to, to less than or less than or equal to and the determination of when the index value indicates pain in the user may be similarly inverted (e.g., not satisfying the threshold indicates pain in the user and satisfying the threshold indicates no pain in the user) in certain examples.

At step 820, an indication of pain of the user 101 may be output. For example, the indication may be output by the computing device 105, the user device 104A or any other device described herein. For example, the output of the indication of pain may cause a display of the indication of pain on a display associated with the computing device 105, the user device 104A, or any other user device described herein. As another example, the indication of pain may be sent from the user device 104A to the computing device 105 or to one of the other user devices 104B-C. For example, the indication, or a signal referencing the indication of the pain, that is output may be sent to a second computing device, such as the computing device 120. For example, the indication of pain that is output may be sent from the computing device 105 to one of the user devices 104A-C. For example, the indication of pain that is caused to be displayed may be based on the value of the index value, or difference in the values of the index values, satisfying the threshold. For example, the indication of pain that is output may comprise a score or pain level for the user 101. For example, the score or pain level for the user 101 may be based on the value of the index value, or difference in the values of the index values, satisfying one or more of a plurality of pain level thresholds. For example, the indication of pain that is output may comprise a source or location of the pain within the user 101.

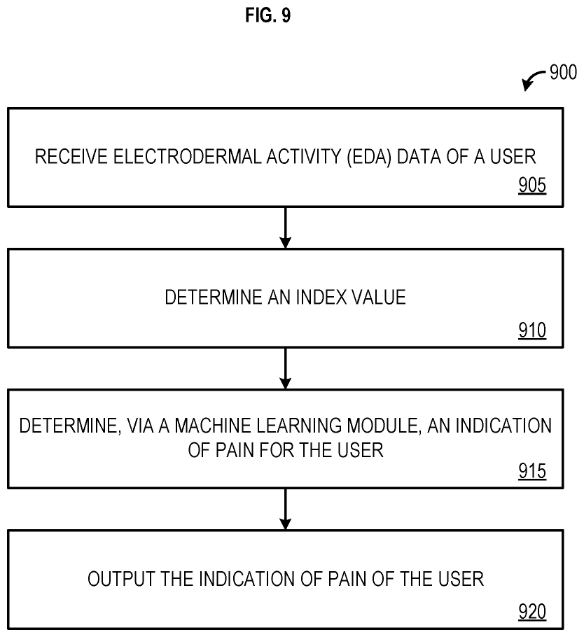

FIG. 9 shows a flowchart of an example method 900 for the indication of pain of a user (e.g., a patient, a drug-dependent individual, a health-compromised individual, or a communication-deficient being). The method 900 may be performed by the computing device 105 and/or any of the user devices 104A-C.

At step 905, EDA data of a user (e.g., the user 101) may be received. For example, the EDA data may be received by the computing device 105, the user device 104A or any other device described herein. For example, the EDA data may be received from one or more sensors, such as the EDA sensor(s) 102 of FIG. 1. For example, the EDA data may be received while the user 101 is experiencing varying instances of pain. For example, EDA data received from the user 101 may be based on varying stimuli associated with pain. As an example, the EDA data may be continuously received in the instance wherein the user 101 selects a constant EDA monitoring option of the user's 101 body (e.g., a smart watch or an at-home patient monitoring device). As an example, the EDA data may be or may be associated with information that may be indicative of one or more physiological signals of the user 101. The one or more physiological signals may be derived from sweat gland activity of the user 101 and received from the EDA sensor(s) 102, or probes associated with the sensors 102, contacting the skin of the user 101.

For example, other sensor data associated with the user 101 may be received. The other sensor data may include one or more of electromyographical data readings of the user 101, electrocardiographical data readings of the user 101, and/or accelerometer data readings associated with movement of the user 101. The electromyographical data readings may be received from one or more electromyography sensor(s), the electrocardiographical data readings may be received from one or more electrocardiography sensor(s), and the accelerometer data may be received from an accelerometer 103. For example, the accelerometer 103 may be a tri-axial accelerometer. For example, the accelerometer may determine movement of the user 101. For example, the movement data of the user 101 may be used to determine portions of the EDA data which may not be desirable to use due to the potential for the motion of the user 101 causing erroneous index values (e.g., TVSymp, MTVSymp, or dPhEDA). Each of the electromyography sensor, the electrocardiography sensor, and the accelerometer 103 may be included with the same device (e.g., the user device 104A) as the one or more EDA sensors 102 or may be part of and received by another device, such as another wearable device or another one of the user devices 104B-C. Each of the EDA data, the accelerometer data, the electromyographical data, and the electrocardiographical data may be received via a wired or wireless (e.g., Bluetooth®, BLE, RF (e.g., electromagnetic RF), WiFi, or any other known wireless communication protocol) communication. For example, EDA data and any of the other data may be sent by the user device 104A. For example, the EDA data and the other data may be sent by the user device 104A as part of an internal transmission from the respective sensors to another portion of the user device 104A. In another example, the user device 104A may send the EDA data and the other sensor data to the computing device 105 or another user device 104B-C via the wired or wireless communication.

A determination may be made as to whether all or portions of the received EDA data is valid. For example, the determination may be made by the computing device 105, the user device 104A or any other device described herein. For example, EDA data received while the user 101 is performing a certain amount of movement or exertion may not be useful in the analysis. For example, accelerometer data may be received from an accelerometer 103. For example, the accelerometer may be associated with the movement of the user 101. The accelerometer data may be compared to a motion threshold. A determination may be made as to if all or portions of the accelerometer data satisfy (e.g., is less than or less than or equal to or is greater than or greater than or equal to) the motion threshold. For example, if a first portion of the accelerometer data does not satisfy (e.g., the accelerometer data is less than or less than or equal to) the motion threshold, a determination may be made that the EDA data associated with (e.g., collected at the same time as) the first portion of the accelerometer data is valid EDA data. For example, if a second portion of the accelerometer data satisfies (e.g., the accelerometer data is greater than or greater than or equal to) the motion threshold, a determination may be made that the EDA data associated with the second portion of the accelerometer data is invalid EDA data. For example, satisfying the motion threshold may be switched from greater than or greater than or equal to, to less than or less than or equal to and the determination of which EDA data is valid and invalid may be similarly inverted (e.g., valid data satisfies the threshold and invalid data does not satisfy the threshold) in certain examples. For example, the second portion of the EDA data comprising invalid data may be removed from further analysis and the first portion of the EDA data comprising valid data may be further analyzed and processed.

At step 910, an index value may be determined. For example, the index value may be determined by the computing device 105, the user device 104A or any other device described herein. For example, the index value may be determined based on the EDA data. For example, the index value may be determined based on the valid EDA data (e.g., the first portion of the EDA data). For example, the index value may be one or more of TVSymp of the EDA data, MTVSymp of the EDA data, or dPhEDA data. The index value may be determined using a frequency complex demodulation, for example. For example, the one or more index values may serve as a basis by which pain occurring in the user 101 is determined.

At step 915, an indication of pain of the user 101 may be determined. For example, the indication of pain may be determined by the computing device 105, the user device 104A or any other device described herein. For example, a determination may be made with regard to whether the index value satisfies a threshold. The indication of pain may be determined via a machine learning module. The machine learning module may be a component of the computing device 105 or any of the user devices 104A-C, for example.

The determination of the indication of pain for the user 101 may be based on the index value satisfying the threshold, for example. For example, a second index value may be derived from a portion of the EDA data. For example, the second index value may be determined based on the EDA data. As another example, the second index value may precede the second portion of the EDA data that the index value may be derived from. For example, whether a difference between the index value and the second index value satisfies a threshold may be determined. As another example, the indication of pain for the user 101 may be based on the difference satisfying the threshold.

The machine learning module may train recorded data sets (e.g., EDA data) based on variables directed toward at least accuracy, sensitivity, and/or specificity. The variables may be implemented and enhanced based on historical data associated with the user 101. The historical data associated with the user 101 may include a pain threshold of the user, for example. For example, a mean is computed across a plurality of users experiencing a similar ailment. The mean may indicate an objective score referencing a pain level. As another example, the mean may be a dynamic variable that may change based on increased readings of various other users, over time.

Various machine learning techniques may be implemented. For example, a regressive machine learning technique may be implemented that may directly estimate a pain level of the user 101. In the instance wherein the pain level of the user 101 is directly estimated, a score may be assigned to the user 101 (e.g., pain levels 0-10). As another example, a machine learning technique directed toward classification of a pain level may be implemented. In the instance wherein the pain level of the user 101 may be identified via a classification method, a calculation of a probability that the pain level of the user 101 may be classified within a particular pain level group (e.g., low, medium, and high level pain groups) may occur. As an example, the classification of the pain level of the user 101 may be categorized by any metric associated with the pain level of the user 101. For example, the metric may be a numerical rating, a pain scale, a source of pain and/or a source of pain associated with a particular ailment.

At step 920, an indication of pain of the user 101 may be output. For example, the indication may be output by the computing device 105, the user device 104A or any other device described herein. For example, the output of the indication of pain may cause a display of the indication of pain on a display associated with the computing device 105, the user device 104A, or any other user device described herein. As another example, the indication of pain may be sent from the user device 104A to the computing device 105 or to one of the other user devices 104B-C. For example, the indication, or a signal referencing the indication of the pain, that is output may be sent to a second computing device, such as the computing device 120. For example, the indication of pain that is output may be sent from the computing device 105 to one of the user devices 104A-C. For example, the indication of pain that is caused to be displayed may be based on the value of the index value, or difference in the values of the index values, satisfying the threshold. For example, the indication of pain that is output may comprise a score or pain level for the user 101. For example, the score or pain level for the user 101 may be based on the value of the index value, or difference in the values of the index values, satisfying one or more of a plurality of pain level thresholds. For example, the indication of pain that is output may comprise a source or location of the pain within the user 101.

While the methods and systems have been described in connection with preferred embodiments and specific examples, it is not intended that the scope be limited to the particular embodiments set forth, as the embodiments herein are intended in all respects to be illustrative rather than restrictive.

While specific configurations have been described, it is not intended that the scope be limited to the particular configurations set forth, as the configurations herein are intended in all respects to be possible configurations rather than restrictive.

Unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is in no way intended that an order be inferred, in any respect. This holds for any possible non-express basis for interpretation, including: matters of logic with respect to arrangement of steps or operational flow; plain meaning derived from grammatical organization or punctuation; the number or type of embodiments described in the specification.

It will be apparent to those skilled in the art that various modifications and variations can be made without departing from the scope or spirit. Other embodiments will be apparent to those skilled in the art from consideration of the specification and practice disclosed herein. It is intended that the specification and examples be considered with only a true scope and spirit being indicated by the following claims.

What is claimed is:

1. A method comprising:
receiving, by a computing device from a sensor, electrodermal activity (EDA) data of a user;
determining, based on the EDA data, an index value;
determining, based on the index value, an indication of pain for the user; and
outputting the indication of pain for the user.

2. The method of claim 1, wherein the index value comprises a time-varying index of sympathetic activity (TVSymp) value of the EDA data, a modified TVSymp (MTVSymp) value of the EDA data, or a derivative of a phasic component of EDA (dPhEDA) value of the EDA data.

3. The method of claim 1, wherein determining the indication of pain for the user comprises:
determining that the index value satisfies a threshold; and
determining, based on the index value satisfying the threshold, the indication of pain for the user.

4. The method of claim 1, wherein determining the indication of pain for the user comprises:
determining, based on the EDA data, a second index value, wherein the second index value is derived from a portion of the EDA data that precedes a second portion of the EDA data that the index value is derived from;
determining a difference between the index value and the second index value satisfies a threshold; and
determining, based on the difference satisfying the threshold, the indication of pain for the user.

5. The method of claim 1, further comprising determining, based on the index value, a level of pain for the user.

6. The method of claim 1, further comprising determining, based on the index value a source of the pain on a body of the user.

7. The method of claim 1, wherein outputting the indication of pain for the user comprises one or more of displaying the indication of pain on a display associated with a user device or sending the indication of pain to the computing device.

8. The method of claim 1, further comprising:
determining a first portion of the EDA data comprises valid data and a second portion of the EDA data comprises invalid data,
wherein determining, based on the EDA data, the index value comprises determining, based on the first portion of the EDA data, the index value.

9. The method of claim 8, further comprising:
receiving, by the computing device, accelerometer data for the user; and
determining, at least a portion of the accelerometer data satisfies a motion threshold, wherein the at least the portion of the accelerometer data corresponds to at least a portion of the second portion of the EDA data,
wherein determining the second portion of the EDA data comprises the invalid data comprises determining, based on the at least the portion of the accelerometer data satisfying the motion threshold, the second portion of the EDA data comprises the invalid data.

10. A method comprising:
receiving, by a computing device from a sensor, electrodermal activity (EDA) data of a user;
determining, based on the EDA data, an index value;
determining, via a machine learning module and based on the index value, an indication of pain for the user; and
outputting the indication of pain for the user.

11. The method of claim 10, wherein the index value comprises a time-varying index of sympathetic activity (TVSymp) value, a modified TVSymp (MTVSymp) value, or a derivative of phasic component of EDA (dPhEDA) value.

12. The method of claim 10, further comprising training, based on historical data of one or more users, the machine learning module to determine the indication of pain for the user.

13. The method of claim 10, wherein determining the indication of pain for the user comprises:
determining that the index value satisfies a threshold; and
determining, based on the index value satisfying the threshold, the indication of pain for the user.

14. The method of claim 10, wherein determining the indication of pain for the user comprises:
determining, based on the EDA data, a second index value, wherein the second index value is derived from a portion of the EDA data that precedes a second portion of the EDA data that the index value is derived from;
determining a difference between the index value and the second index value satisfies a threshold; and
determining, based on the difference satisfying the threshold, the indication of pain for the user.

15. The method of claim 10, further comprising determining, based on the index value, a level of pain for the user.

16. The method of claim 10, further comprising determining, based on the index value a source of the pain on a body of the user.

17. The method of claim 10, wherein outputting the indication of pain for the user comprises one or more of displaying the indication of pain on a display associated with a user device or sending the indication of pain to the computing device.

18. The method of claim 10, further comprising:
determining a first portion of the EDA data comprises valid data and a second portion of the EDA data comprises invalid data,
wherein determining, based on the EDA data, the index value comprises determining, based on the first portion of the EDA data, the index value.

19. The method of claim 18, further comprising:
receiving, by the computing device, accelerometer data for the user; and
determining, at least a portion of the accelerometer data satisfies a motion threshold, wherein the at least the portion of the accelerometer data corresponds to at least a portion of the second portion of the EDA data,
wherein determining the second portion of the EDA data comprises the invalid data comprises determining, based on the at least the portion of the accelerometer data satisfying the motion threshold, the second portion of the EDA data comprises the invalid data.

20. An apparatus comprising:
one or more processors; and
a memory storing processor-executable instructions that, when executed by the one or more processors, cause the apparatus to:
receive, by a computing device from a sensor, electrodermal activity (EDA) data of a user;
determine, based on the EDA data, an index value;
determine, based on the index value, an indication of pain for the user; and
output the indication of pain for the user.
